(19) United States
(12) Patent Application Publication
(10) Pub. No.: US 2013/0110091 A1
(43) Pub. Date: May 2, 2013

(54) APPARATUS AND METHOD FOR PERFORMING SURGICAL EYE PROCEDURES INCLUDING LTK AND CXL PROCEDURES

(71) Applicant: NTK Enterprises, Inc., Plano, TX (US)

(72) Inventor: Michael J. Berry, Austin, TX (US)

(73) Assignee: NTK Enterprises, Inc., Plano, TX (US)

(21) Appl. No.: 13/647,802

(22) Filed: Oct. 9, 2012

Related U.S. Application Data
(60) Provisional application No. 61/551,789, filed on Oct. 26, 2011.

(51) Int. Cl. A61B 18/20

(52) U.S. Cl. 606/3; 606/5

(57) ABSTRACT

A system includes at least one radiation source configured to generate radiation for a cornea reshaping procedure and a corneal cross-linking procedure. The system also includes a delivery device configured to deliver the radiation to the patient's eye. The system further includes a controller configured to control the delivery of the radiation to the patient's eye during the cornea reshaping procedure and the corneal cross-linking procedure. The controller may be configured to control the delivery of radiation so that the cornea reshaping procedure and the corneal cross-linking procedure at least partially overlap in time. The delivery device may be configured to deliver radiation to specified areas of the patient's eye during the cornea reshaping procedure and to deliver radiation only to the same specified areas of the patient's eye during the corneal cross-linking procedure.

### CROSS-REFERENCE TO RELATED APPLICATIONS

This application claims priority under 35 U.S.C. §119(e) to U.S. Provisional Patent Application No. 61/551,789 filed on Oct. 26, 2011.

This application is also related to the following patent documents:

U.S. patent application Ser. No. 11/440,794 filed on May 25, 2006;

U.S. patent application Ser. No. 11/825,816 filed on Jul. 9, 2007 (now U.S. Pat. No. 7,691,099); and

U.S. patent application Ser. No. 12/191,784 filed on Aug. 14, 2008.

All four of these patent documents are hereby incorporated by reference.

### TECHNICAL FIELD

This disclosure is generally directed to cornea reshaping. More specifically, this disclosure is directed to an apparatus and method for performing surgical eye procedures, including laser thermal keratoplasty (LTK) and corneal cross-linking (CXL) procedures.

### BACKGROUND

Today, there are hundreds of millions of people in the United States and around the world who wear eyeglasses or contact lenses to correct ocular refractive errors. The most common ocular refractive errors include myopia (nearsightedness), hyperopia (farsightedness), astigmatism, and presbyopia. Each of these ocular refractive errors can be modified, reduced, or corrected by reshaping the cornea of a patient's eye.

Various procedures have been used to correct ocular refractive errors. For example, laser thermal keratoplasty (LTK) uses laser light to heat the cornea, which causes shape changes in the cornea. As another example, corneal cross-linking (CXL) is an attractive procedure for reducing the progression of keratoconus (and hence undesirable corneal shape change) by increasing corneal biomechanical strength. The CXL procedure uses a photo-sensitizer such as riboflavin together with ultraviolet or visible light to produce cross-links in corneal tissue that increase the stiffness and strength of the corneal tissue (compared to natural stromal corneal tissue). Other surgical eye procedures may use radio frequency signals, laser irradiation, or other techniques to treat the corneal tissue in a patient's eye.

### SUMMARY

This disclosure provides an apparatus and method for performing surgical eye procedures, including laser thermal keratoplasty (LTK) and corneal cross-linking (CXL) procedures.

In a first embodiment, a system includes at least one radiation source configured to generate radiation for a cornea reshaping procedure and a corneal cross-linking procedure. The system also includes a delivery device configured to deliver the radiation to the patient's eye. The system further includes a controller configured to control the delivery of the radiation to the patient's eye during the cornea reshaping procedure and the corneal cross-linking procedure.

In a second embodiment, an apparatus includes at least one memory configured to store treatment parameters for a cornea reshaping procedure and a corneal cross-linking procedure. The apparatus also includes at least one processing device configured to control at least one radiation source in order to control delivery of radiation to a patient's eye during the cornea reshaping procedure and the corneal cross-linking procedure.

In a third embodiment, a method includes generating radiation for a cornea reshaping procedure and a corneal cross-linking procedure. The method also includes delivering the radiation to a patient's eye and controlling the delivery of the radiation to the patient's eye during the cornea reshaping procedure and the corneal cross-linking procedure.

In a fourth embodiment, an optical device includes an optical surface having alternating angular regions of greater and lesser refractive powers.

Other technical features may be readily apparent to one skilled in the art from the following figures, descriptions, and claims.

### BRIEF DESCRIPTION OF THE DRAWINGS

For a more complete understanding of this disclosure and its features, reference is now made to the following description, taken in conjunction with the accompanying drawings, in which:

FIG. 1 illustrates an example system for cornea reshaping and other surgical eye procedures according to this disclosure;

FIG. 2 illustrates an example controller for cornea reshaping and other surgical eye procedures according to this disclosure;

FIGS. 3A and 3B illustrate examples of an extracellular matrix structure of an eye and effects of cornea reshaping or other surgical eye procedure on the eye according to this disclosure;

FIGS. 4A through 4G illustrate example temperature graphs showing how surgical eye procedures may or may not produce corneal tissue heating according to this disclosure;

FIG. 5 illustrates an example method for designing and implementing a cornea reshaping procedure according to this disclosure;

FIGS. 6 through 9 illustrate example details regarding a corneal cross-linking (CXL) procedure according to this disclosure;

FIGS. 10 through 12 illustrate example multi-focal refraction patterns that can be used during a CXL or other procedure or in optical devices according to this disclosure; and

FIG. 13 illustrates an example method for designing and implementing a cornea reshaping procedure involving LTK and CXL according to this disclosure.

### DETAILED DESCRIPTION

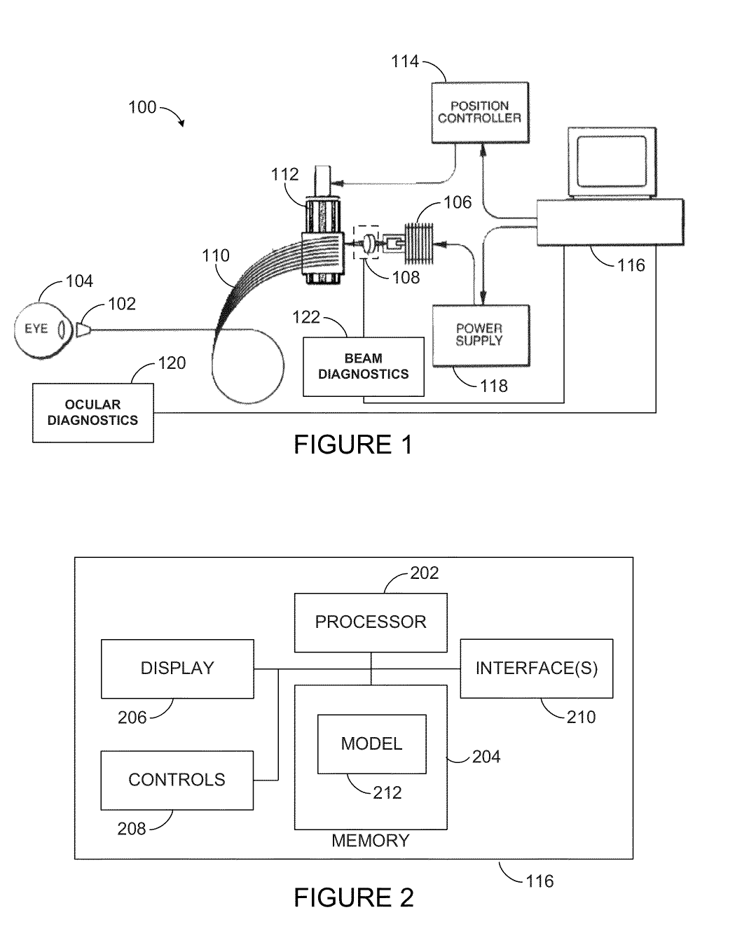

FIGS. 1 through 12, discussed below, and the various embodiments used to describe the principles of the present invention in this patent document are by way of illustration only and should not be construed in any way to limit the scope of the invention. Those skilled in the art will understand that the principles of the invention may be implemented in any type of suitably arranged device or system.

FIG. 1 illustrates an example system 100 for cornea reshaping and other surgical eye procedures according to this disclosure. The embodiment of the system 100 shown in FIG. 1 is for illustration only. Other embodiments of the system 100 may be used without departing from the scope of this disclosure.

In this example, the system 100 includes a protective corneal applanator device 102. The protective corneal applanator device 102 is pressed against a patient's eye 104 during a cornea reshaping procedure. For example, the protective corneal applanator device 102 may be used during a laser thermal keratoplasty (LTK) procedure, a corneal cross-linking (CXL) procedure, or other procedure meant to treat one or more conditions of the eye 104.

Among other things, the protective corneal applanator device 102 helps to reduce or eliminate damage to the corneal epithelium of the patient's eye 104 during a surgical eye procedure. For example, the protective corneal applanator device 102 could act as a heat sink to conduct heat away from the patient's eye 104 during a procedure. This helps to reduce the temperature of the corneal epithelium, which may help to reduce or eliminate damage to the corneal epithelium and avoid a corneal wound healing response that could lead to regression of refractive correction. Example embodiments of the protective corneal applanator device 102 are disclosed in U.S. patent application Ser. No. 11/440,794 filed on May 25, 2006 and in U.S. patent application Ser. No. 11/825,816 filed on Jul. 9, 2007, which are both hereby incorporated by reference. In this document, the phrase “cornea reshaping procedure” refers to any procedure involving a patient's eye 104 that results in a reshaping of the cornea in the eye 104, whether the reshaping occurs immediately or over time. Also, the phrase “surgical eye procedure” refers to any procedure involving a patient's eye 104 that involves surgical treatment of the cornea in the eye 104.

The system 100 also includes one or more treatment light sources 106. Light from the treatment light sources 106 is used to irradiate the patient's eye 104 during a surgical eye procedure. The treatment light sources 106 represent any suitable sources capable of providing light for a surgical eye procedure, such as a laser, infrared, or near-infrared source for LTK procedures and an ultraviolet or visible light source for CXL procedures. Example lasers that could be used include a continuous wave laser (such as a continuous wave hydrogen fluoride chemical laser or a continuous wave thulium fiber laser) or a pulsed laser (such as a pulsed holmium:yttrium aluminum garnet or “Ho:YAG” laser). Any other suitable laser or non-laser light source capable of providing suitable radiation for a surgical eye procedure could also be used in the system 100. Example light sources for CXL procedures can include semiconductor diode lasers that produce ultraviolet or visible light at 370 nm, 395 nm, 405 nm, or other wavelength(s).

The light produced by the treatment light sources 106 is provided to a beam distribution system 108. The beam distribution system 108 focuses the light from the treatment light sources 106. For example, the beam distribution system 108 could include optics that focus the light from the treatment light sources 106 to control the geometry, dose, and irradiance level of the light as it is applied to the cornea of the patient's eye 104 through a fiber optic array 110 during a surgical eye procedure. The beam distribution system 108 could also include a shutter for providing a correct exposure duration of the light. In addition, the beam distribution system 108 could include a beam splitting system for splitting the focused light into multiple beams (which may be referred to as “beams,” “treatment beams,” or “beamlets”). The beam distribution system 108 includes any suitable optics, shutters, splitters, or other or additional structures for generating one or more beams for a surgical eye procedure. Examples of the beam splitting system in the beam distribution system 108 are disclosed in various U.S. patent applications incorporated by reference above.

One or more beams from the beam distribution system 108 are transported to the protective corneal applanator device 102 using the fiber optic array 110. The fiber optic array 110 includes any suitable structure(s) for transporting one or multiple laser beams or other light energy to the protective corneal applanator device 102. The fiber optic array 110 could, for example, include multiple groups of fiber optic cables, such as groups containing four fiber optic cables each. The fiber optic array 110 could also include attenuators that adjust fiber outputs so as to change optical fiber transmission through the array 110.

A translation stage 112 moves the fiber optic array 110 so that light from the treatment light sources 106 enters different ones of the fiber optic cables in the fiber optic array 110. For example, the beam distribution system 108 could produce four beams, and the translation stage 112 could move the fiber optic array 110 so that the four beams enter different groups of four fiber optic cables. Different fiber optic cables could deliver light onto different areas of the cornea in the patient's eye 104. The translation stage 112 allows the different areas of the cornea to be irradiated by controlling which fiber optic cables are used to transport the beams from the beam distribution system 108 to the protective corneal applanator device 102. The translation stage 112 includes any suitable structure for moving a fiber optic array. While the use of four beams and groups of four fiber optic cables has been described, any suitable number of beams and any suitable number of fiber optic cables could be used in the system 100.

A position controller 114 controls the operation of the translation stage 112. For example, the position controller 114 could cause the translation stage 112 to translate, thereby repositioning the fiber optic array 110 so that beams from the beam distribution system 108 enter a different set of fiber optic cables in the array 110. The position controller 114 includes any hardware, software, firmware, or combination thereof for controlling the positioning of a fiber optic array.

A controller 116 controls the overall operation of the system 100. For example, the controller 116 could ensure that the system 100 provides predetermined patterns and doses of light onto the anterior surface of the cornea in the patient's eye 104. This allows the controller 116 to ensure that an LTK, CXL, or other procedure is carried out properly on the patient's eye 104. In some embodiments, the controller 116 includes all of the controls necessary for a surgeon or other physician to have complete control of a surgical eye procedure, including suitable displays of operating variables showing what parameters have been preselected and what parameters have actually been used. As a particular example, the controller 116 could allow a surgeon to select, approve of, or monitor a pattern of irradiation of the patient's eye 104. If a pulsed light source 106 is used, the controller 116 could also allow the surgeon to select, approve of, or monitor the pulse duration, the number of pulses to be delivered, the number of pulses actually delivered to a particular location on the patient's eye 104, and the irradiance of each pulse. Moreover, the controller 116 may synchronize the actions of various components in the system 100 to obtain accurate delivery of light onto the cornea of the patient's eye 104. In addition, as described in more detail below, the controller 116 could design and then implement an LTK or other surgical eye procedure, where the surgical eye procedure is designed so that the desired corneal shape changes occur with little or no stromal collagen shrinkage in the patient's eye.

The controller 116 includes any hardware, software, firmware, or combination thereof for controlling the operation of the system 100. As an example, the controller 116 could represent a computer (such as a desktop or laptop computer) at a surgeon's location capable of displaying elements of a surgical eye procedure that are or may be of interest to the surgeon. An example embodiment of the controller 116 is shown in FIG. 2, which is described below.

A power supply 118 provides power to the treatment light sources 106. The power supply 118 is also controlled by the controller 116. This allows the controller 116 to control if and when power is provided to the treatment light sources 106. The power supply 118 represents any suitable source(s) of power for the treatment light sources 106.

As shown in FIG. 1, the system 100 also includes one or more ocular diagnostic tools 120. The ocular diagnostic tools 120 may be used to monitor the condition of the patient's eye 104 before, during, and/or after a surgical eye procedure. For example, the ocular diagnostic tools 120 could include a keratometer or other corneal topography measuring device, which is used to measure the shape of the cornea in the patient's eye 104. By comparing the shape of the cornea before and after the procedure, this tool may be used to determine a change in the shape of the cornea. After treatment, keratometric measurements may be performed to produce corneal topographic maps that verify the desired correction has been obtained. In some embodiments, the keratometer may provide a digitized output from which a visual display is producible to show the anterior surface shape of the cornea. As another example, the ocular diagnostic tools 120 could include a mechanism for viewing the cornea in the patient's eye 104 during the procedure, such as a surgical microscope or a slit-lamp biomicroscope. Any other or additional ocular diagnostic tools 120 could be used in the system 100.

In addition, the system 100 may include a beam diagnostic tool 122. The beam distribution system 108 could include a beam splitter that samples a small portion (such as a few percent) of one or more light beams. A sampled beam could represent the beam that is to be split or one of the beams after splitting. The sampled portion of the beam is directed to the beam diagnostic tool 122, which measures beam parameters such as power, spot size, and irradiance distribution. In this way, the controller 116 can verify whether the patient's eye 104 is receiving a proper amount of treatment light and whether various components in the system 100 are operating properly.

In one aspect of operation, a patient may lie down on a table that includes a head mount for accurate positioning of the patient's head. The protective corneal applanator device 102 may be attached to an articulated arm that holds the device 102 in place. The articulated arm may be attached to a stable platform, thereby helping to restrain the patient's eye 104 in place when the protective corneal applanator device 102 is attached to the patient's eye 104. The patient may look up toward the ceiling during the procedure, and beams transported by the fiber optic array 110 may be directed vertically downward onto the patient's eye 104. Other procedures may vary from this example. For example, the protective corneal applanator device 102 may have a small permanent magnet mounted on the center of its front surface. This magnet may be used to attach and centrate a fiber optic holder shaft on the protective corneal applanator device 102 using another small permanent magnet that is mounted on the fiber optic holder shaft.

A surgeon or other physician who performs the cornea reshaping procedure may use a tool (such as an ophthalmic surgical microscope, a slit-lamp biomicroscope, or other tool 120), together with one or more visible tracer laser beams (from a low energy visible laser such as a helium-neon laser) collinear with the treatment beams, to verify the proper positioning of the treatment beams. The surgeon or other physician also uses the controller 116 to control the system 100 so as to produce the correct pattern, irradiance, and exposure duration of the treatment beams. The controller 116 could be used by the surgeon or other physician as the focal point for controlling all variables and components in the system 100. During the procedure, the treatment light sources 106 produce functionally effective light, which is processed to produce the correct pattern and dose of functionally effective light on the anterior surface of the cornea in the patient's eye 104.

As described in more detail below, the controller 116 can also design the LTK, CXL, or other surgical eye procedure before the surgical eye procedure occurs. For example, the controller 116 could select the appropriate geometry, dose, irradiance level, exposure duration, or any other or additional parameters for a surgical eye procedure. The surgical eye procedure can be designed so that the desired corneal shape changes to the patient's eye occur with little or no stromal collagen shrinkage in the patient's eye.

Although FIG. 1 illustrates one example of a system 100 for cornea reshaping and other surgical eye procedures, various changes may be made to FIG. 1. For example, while FIG. 1 illustrates a system for irradiating a patient's eye 104 using multiple beams transported over a fiber optic array 110, the system 100 could generate any number of beams (including a single beam) for irradiating the patient's eye 104. Also, various components in FIG. 1 could be combined or omitted and additional components could be added according to particular needs, such as by combining the controllers 114, 116 into a single functional unit. In addition, other techniques could be used to treat corneal tissue of a patient's eye, instead of or in addition to irradiation using lasers or ultraviolet/visible light (such as radio frequency heating).

FIG. 2 illustrates an example controller 116 for cornea reshaping and other surgical eye procedures according to this disclosure. The embodiment of the controller 116 shown in FIG. 2 is for illustration only. Other embodiments of the controller 116 could be used without departing from the scope of this disclosure.

In this example, the controller 116 includes at least one processor 202, at least one memory 204, at least one display 206, controls 208, and at least one interface 210. The processor 202 represents any suitable processor or processing device for executing instructions that implement the functionality of the controller 116. The processor 202 could, for example, represent a microprocessor, microcontroller, or other suitable processor or processing device. The memory 204 stores instructions and data used, generated, or collected by the processor 202. The memory 204 includes any suitable volatile and/or non-volatile storage and retrieval device or devices, such as a RAM, ROM, EPROM, EEPROM, or flash memory.

The display 206 presents information to a user, such as parameters related to a selected surgical eye procedure. The display 206 includes any suitable structure for presenting information, such as a liquid crystal display. The controls 208 are used to control the operation of the controller 116. The controls 208 could, for example, include controls allowing a surgeon or other personnel to accept, adjust, or reject parameters for a surgical eye procedure. Any suitable controls 208 could be used, such as dials, buttons, keypad, or keyboard. The interface 210 includes any suitable structure facilitating communication with an external device or system, such as the position controller 114, power supply 118, or beam distribution system 108.

As described above, the controller 116 could be capable of designing and then implementing an LTK, CXL, or other surgical eye procedure. Moreover, the controller 116 could design the surgical eye procedure such that any desired corneal shape changes occur with little or no stromal collagen shrinkage. As noted above, it has long been believed that LTK and other surgical eye procedures modified the shape of the cornea due to shrinkage of stromal collagen in the eye (which was caused by laser irradiation or other form of heating). As a result, LTK and other surgical eye procedures were based on the idea that laser heating produces thermal shrinkage of stromal collagen in the eye.

Recent research and clinical results, however, point to discrepancies between observations and expectations derived from a “standard” model of LTK and actual real-world results. Based on this, stromal collagen shrinkage may have little or no contribution to the correction of ocular refractive errors under optimal conditions and may actually be undesirable. The phrase “optimal conditions” may refer to any treatment conditions that cause a desired or predetermined corneal shape change without causing a significant fibrotic wound healing response, significant opacification, and significant stromal collagen shrinkage.

The actual kinetics of corneal collagen shrinkage have been measured by Borja et al. (2004), and the kinetic results were analyzed in terms of two first-order rate coefficients k₁ and k₂ (corresponding to two “lifetimes” τ₁=k₁⁻¹ and τ₂=k₂⁻¹) that pertain to two processes: shrinkage during heating from a starting physiological temperature of approximately 33° C. (or some other starting temperature such as a room temperature of approximately 20° C.) to a “target temperature” (rate 1) followed by regression after cooling from the “target temperature” to the starting temperature (rate 2) (note that these values may vary). The shrinkage rate coefficient k₁ is a function of temperature and is represented by an Arrhenius equation:

k₁(T)=Aexp(−Eₐ/RT)

where A is the pre-exponential factor (units: s⁻¹), Eₐ is the activation energy (units: kJ mol⁻¹), R is the gas constant (8.314 J K⁻¹ mol⁻¹), and T is the temperature (units: K). In some embodiments, over a range of T=60 to 80° C., A=8.96×10¹⁴ s⁻¹ and Eₐ=112.8 kJ mol⁻¹. At the highest temperatures used (approximately 80 to 90° C.), the rate coefficient was measured to be approximately k₁=0.014 to 0.015 s⁻¹.

In view of this, under optimal conditions (such as using a protective corneal applanator device 102 with a starting temperature of approximately 20° C. and using a continuous wave thulium fiber laser operating at a wavelength of 1.94 μm to deliver a maximum energy density of 40 mJ/spot, 48 mJ/spot, or other energy density with a spot size of approximately 600 μm diameter with nearly uniform irradiance distribution over the spot for an irradiation time of 0.15 s), a small amount of collagen (such as less than 1% or less than 5% of the collagen within the irradiated spot) can be shrunk. Using the same optimal conditions but less laser irradiation at a minimum energy density of 30 mJ/spot (typically used in LTK and other procedures), an even smaller amount of collagen (such as less than 0.01% of the collagen within the irradiated spot) can be shrunk. If these small amounts of collagen were shrunk within each irradiated spot, it should be possible to irradiate the same spot multiple times to shrink more collagen and produce a larger cumulative effect. However, experiments have shown that multiple irradiations of the same spot do not produce significantly more keratometric changes than a single irradiation. This observation, together with the above estimates based on measured kinetics of collagen shrinkage, indicates that stromal collagen shrinkage may not be a major mechanism of action in LTK and other procedures.

Moreover, opacifications (i.e. light-scattering or reduced transparency volumes of corneal tissue) are typically observed in irradiated spots in a patient's eye. Opacifications typically fade as a function of time, such as one to two years following a surgical eye procedure. However, long-term LTK effects may often persist, while opacifications do not. It was assumed that long-term opacification was caused primarily by collagen shrinkage (as opposed to short-term opacification, which could also be caused by stromal hydration changes). As a result, the disappearance of long-term opacification was assumed to correlate with corneal wound healing that caused removal of shrunken collagen and replacement with new collagen. It was therefore expected that corneal shape changes would completely regress if shrunken collagen was removed and replaced with new collagen.

The basis for the assumed connection between shrunken collagen and opacification was the understanding of corneal transparency in which small and uniform collagen fibril diameters, as well as nearly uniform collagen interfibrillar spacings, are key elements in reducing light scattering. If collagen fibrils are denatured (thereby producing collagen shrinkage), they are also enlarged in diameter and randomized in diameter and interfibrillar spacing, leading to increased scattering and loss of corneal transparency (i.e. “opacification”). Opacifications are undesirable since they scatter light, producing optical aberrations such as glare and halo effects. They also correlate with discomfort, such as photophobia and tearing.

In addition, depressions (such as “indentations,” “dings,” or “divots”) are often observed in irradiated spots. If thermal irradiation of stroma produced only collagen shrinkage, a protrusion in each treated spot would be expected. Stromal collagen shrinkage involves contraction along the main (long) axis of collagen fibrils, but swelling would occur perpendicular to the linear fibril direction. Stromal collagen shrinkage would be expected to increase the amount of collagen post-treatment in the original pre-treatment volume since collagen would be “pulled into” the spot from the periphery. The added volume of collagen, together with its swelling, should cause net protrusion.

With this in mind, three conclusions can be reached. First, stromal collagen shrinkage is not a major cause of opacification, tissue depression, or corneal shape changes following LTK and other surgical eye procedures performed under optimal treatment conditions. Second, opacification from any source should be avoided. Third, some other mechanism(s) of action may be primarily responsible for producing the desired corneal shape changes during optimal LTK and other surgical eye procedures. In other words, stromal collagen shrinkage may be a negligible contribution to corneal shape changes, and stromal collagen shrinkage may be undesirable since it may lead to permanent opacification when the epithelium is protected and fibrotic wound healing is prevented (using the protective corneal applanator device 102). In this case, there may be no wound healing to “clean up” opacified spots. Because of this, the other mechanism(s) of action may be used (to the exclusion of stromal collagen shrinkage) in designing an LTK or other surgical eye procedure.

One possible explanation for the corneal changes that occur during LTK and other procedures is that stromal collagen interfibrillar spacing is decreased. This has been tested using polarized light microscopy to examine the birefringence of treated stromal tissue. Stromal collagen (mostly Type I collagen) is an anisotropic molecule that is birefringent, meaning it has different indices of refraction along its long axis and perpendicular to that axis. This is often called “intrinsic” birefringence. Fibrillar collagen also displays “form” birefringence due to the regular ordering of individual collagen molecules in fibrils, which are themselves in a regular order. Both of these sources of birefringence are lost if collagen shrinkage or denaturation occurs. However, Vogel and Thomsen (2005) found increased birefringence at the edges of thermal coagulation lesions produced in corneal tissue by both laser and radio frequency heating.

Within a patient's eye, the stroma is the main structural element of the cornea in which fibrillar collagen is located. It is composed of cells (primarily keratocytes) that may occupy, for example, 9 to 17% of the total stromal volume distributed through an extracellular matrix (ECM). The extracellular matrix is primarily formed of Type I collagen fibrils (although many other collagens may be present) embedded in a gel-like matrix of proteoglycans (PGs), water, and other ECM materials. By weight, the stroma is approximately 78% water and 22% solids.

The corneal stroma also includes collagen fibrils organized into lamellae that stretch from limbus to limbus in the eye. Anterior lamellae may be interwoven in three dimensions. Jester et al. (2008) have used second harmonic generation imaging to show that anterior stromal lamellae appear to insert into Bowman's layer (the acellular region of the anterior stroma to which the epithelial basement membrane is attached) and to run transverse to the anterior corneal surface. These “sutural” lamellae may have significant influence on the anterior surface shape of the cornea. Posterior lamellae may be parallel to the corneal surface, ordered nearly orthogonally, and relatively easy to dissect (similar to onion skin that can be peeled apart layer-by-layer).

The corneal stroma further includes collagen fibrils that have nearly uniform (and small) diameters, as well as short-range order (i.e. not a perfect lattice but more like an ordered liquid) with nearly uniform interfibrillar spacings. Both the fibril diameter and interfibrillar spacing may be regulated by proteoglycans and by hydration control.

The proteoglycans include core proteins that are bound to collagen fibrils (with horseshoe shaped moieties on the core proteins that regulate collagen fibril diameter). The core proteins are attached to anionic glycosaminoglycan (GAG) chains. These GAGs are carbohydrate “bristles” that project from the collagen fibrils and that interconnect (possibly) next-nearest neighbor fibrils. Free GAGs (not bound to core proteins) may also be present and may bind to each other. Anionic GAGs may have a high affinity for water, which may be present in both “bound” and “free” forms in the stroma.

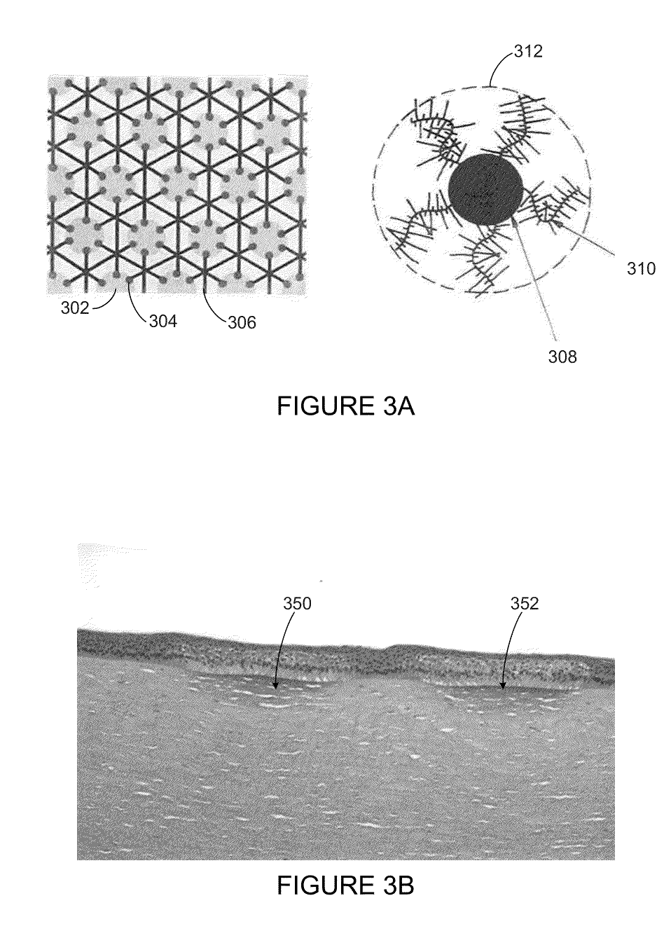

The stromal structure with respect to the network of collagen fibrils bound to proteoglycans is illustrated in FIG. 3A. In particular, FIG. 3A (from Muller et al. (2004) on the left and Fratzl and Daxer (1993) on the right) illustrates the collagen-proteoglycan structure viewed perpendicular to main axes of the collagen fibrils. On the left side of FIG. 3A, collagen fibrils 302 bound to core proteins 304 are connected by GAGs 306. On the right side of FIG. 3A, a single collagen fibril 308 with partly compressed proteoglycans 310 is located inside a unit diameter 312, which is determined by the stromal hydration state. Not shown in FIG. 3A are water molecules that bind to the fibrils 302, 308 and to “bristles” of the proteoglycans 310. Water is the dominant mass fraction (such as approximately 78%) and forms a “gel” in interaction with the fibrils 302 and 308, proteoglycans 310, and other ECM materials.

The above equilibrium stromal structure is perturbed when LTK or other surgical eye procedures are performed. The structural change has been visualized by histology. FIG. 3B illustrates the histology of a cornea following irradiation at a high energy density (60 mJ/spot) with epithelial protection using optimal conditions as described above. Two laser-treated spots 350-352 are shown in FIG. 3B, which are separated at their centers by approximately 0.6 mm.

The treated spots 350-352 are visible at the top of the tissue section as slightly darkened areas of stroma underneath a “stretched” epithelium (where basal epithelial cells are elongated). The anterior surface of the treated stroma is depressed where it meets the basal epithelium. Structural changes other than stromal collagen shrinkage may be causing the depressions in the spots 350-352. One possibility is that the stromal hydration has been decreased. If water is forced out of the treated tissue by heating, the collagen interfibrillar spacing decreases, leading to stromal compression and tissue depression (as is observed in FIG. 3B). Moreover, there appears to be a larger density of “vacuoles” in the treated spots compared to the surrounding tissue. These vacuoles may be associated with collagen fibrillar and lamellar water that has been “expressed” from the tissue into interlamellar spaces. Another possible structural change in the anterior stroma may be the contraction of tissue by modification of the “sutural” lamellae that insert into Bowman's layer of the eye, and this contraction may also be linked to a decrease in stromal hydration.

In general, many thermal modifications other than stromal collagen shrinkage may occur in corneal tissue during irradiation. These include water redistribution, transport of water and/or of nonaqueous materials out of the heated zone, and dissociation of weakly bound complexes. These complexes may include PG-PG, PG-collagen, FACIT-Type I collagen (where FACITs are fibril-associated collagens with interrupted triple helices), and “bound” to “free” water transition. All of these thermally mediated processes may have their own temperature-time histories that are functions of:

irradiation parameters (such as wavelength, irradiance distribution, and irradiation time);

tissue composition (which may vary as a function of position within the stroma);

reaction and transport kinetics;

patient factors (such as age, which may affect protein cross-linking and other variables); and

mechanical loading (such as intraocular pressure).

All of these thermal modifications other than stromal collagen shrinkage can be exploited to design an improved or optimal surgical eye procedure.

In light of this, one or more models 212 of collagen reshaping can be generated and used to design an LTK or other procedure for a particular patient. The models 212 could use the various temperature-time histories and patient factors described above to determine the proper settings for the system 100 (such as irradiation wavelength, irradiation power, spot size, and duration of irradiation).

Each model 212 could represent any suitable mathematical model for predicting or estimating how laser or other irradiation, radio frequency heating, or other treatment of corneal tissue changes the shape of an eye. For example, consider the shape change shown in FIG. 3B. The pre-treatment corneal circumference may be nearly circular. At a specified optical zone (OZ), the pre-treatment circumference c may be defined by c=πd, where d represents the diameter of the optical zone (a centerline ring diameter of 6 mm or 7 mm is used in typical treatments). For the 6 mm optical zone, c=18.85 mm, which is the pre-treatment ring perimeter.

Assume that this perimeter is unchanged in length but is perturbed in shape by the LTK or other treatment due to depressions at eight spots (including spots 350-352) around the ring. The “perturbed” diameter of the post-treatment ring is “cinched” or “tightened” compared to the pre-treatment diameter due to the depressions. As a particular example, in FIG. 3B, stromal depressions in contact with basal epithelial cells appear wavy and could have a maximum depth of approximately 20 μm (although this depth may vary). The basal epithelial cells are “pulled” downward into the depressions. If the surrounding stromal tissue outside the spots is also “pulled” laterally into or towards the depressions, there may be a “belt-tightening” effect, which is often seen under slit-lamp biomicroscope examination as “striae.”

If the lateral displacement of stromal tissue is similar to the axial displacement, each spot may be reduced by approximately 20 μm lateral dimension (in two orthogonal directions, both in the plane of the photomicrograph and also perpendicular to the plane). The “perturbed” diameter may therefore be reduced by approximately 20 μm/spot. For 8 spots, this changes the diameter by approximately 160 μm. At the 6 mm optical zone, the “perturbed” circumference may therefore be equal to approximately 18.7 mm (i.e. a “tighter belt” than the original circumference of 18.85 mm). This perturbation would produce an approximately 1% change in the radius of curvature of the cornea, which translates into approximately 0.4D of corneal steepening. This can be incorporated into a model 212 and used to select the appropriate parameters for a surgical eye procedure.

This example model 212 may be oversimplistic, but it illustrates how a model 212 can be used to design and then implement a surgical eye procedure. More complex models 212 could also be developed and used. For example, stromal lamellae are anisotropic and interwoven in three dimensions in the anterior stroma, and “sutural” lamellae may have significant effects on the anterior cornea surface shape. This means that tissue displacement effects may not be localized. Instead, localized depressions may lead to non-localized displacements elsewhere in the cornea. This non-localized shape change, which may be connected to corneal multi-focality produced by LTK treatment, can be incorporated into the model 212. Also, polarized light microscopy can be used to examine treated spots for increased birefringence and to thereby optimize treatment conditions determined using the models 212 to achieve closer packing of collagen fibrils without collagen shrinkage. In addition, nonlinear microscopy techniques, such as second harmonic generation microscopy, can be used to visualize photothermally-induced disordered corneal collagen in order to optimize laser thermal keratoplasty with little or no collagen shrinking. Additional details of this approach could be found, for example, in Matteini et al., “Photothermally induced disordered patterns of corneal collagen revealed by SHG imaging”, Optics Express 2009, pp. 4868-4878 (which is hereby incorporated by reference).

Moreover, patient-to-patient variability may be overcome by using staged treatments in which the primary treatment produces part of the desired corneal shape change. A particular patient's response to the primary treatment may then be used to plan a secondary treatment that produces most or all of the remaining desired corneal shape change. Further (tertiary and other) treatments may also be used to “titrate” the corneal shape change optimally.

In addition, patient eyes change as they age. For example, progressive loss of accommodation typically occurs, and patients require increasing amounts of magnification (“adds”) for good reading vision. As another example, progressive hyperopic shift as a function of age also occurs in the general population. Since patient eyes typically change as a function of age, it is desirable to use a cornea reshaping or other surgical procedure that can be applied many times over the patient's lifetime without causing “opacifications” and other complications associated with collagen shrinkage.

Although FIG. 2 illustrates one example of a controller 116 for cornea reshaping and other surgical eye procedures, various changes may be made to FIG. 2. For example, the controller 116 could include any other or additional components according to particular needs. Also, while the controller 116 has been described as designing an LTK or other surgical procedure using the model(s) 212, this functionality could be implemented elsewhere (such as on a separate device), and the designed procedure could be provided to the controller 116 for implementation. While FIGS. 3A and 3B illustrate examples of an extracellular matrix structure of an eye and effects of a cornea reshaping or other surgical eye procedure on the eye, the structure and effects may vary, such as from patient to patient or treatment to treatment.

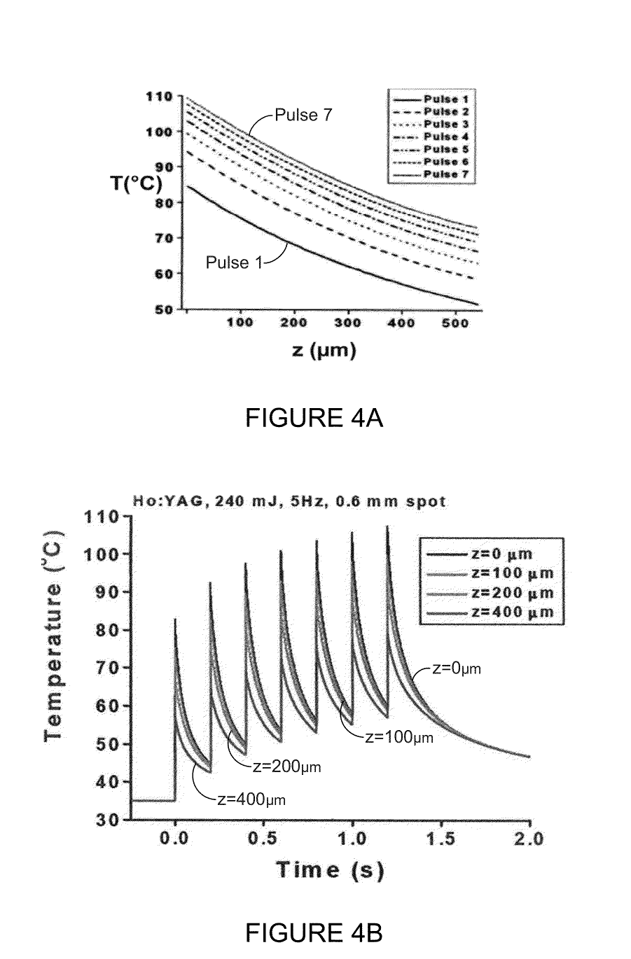

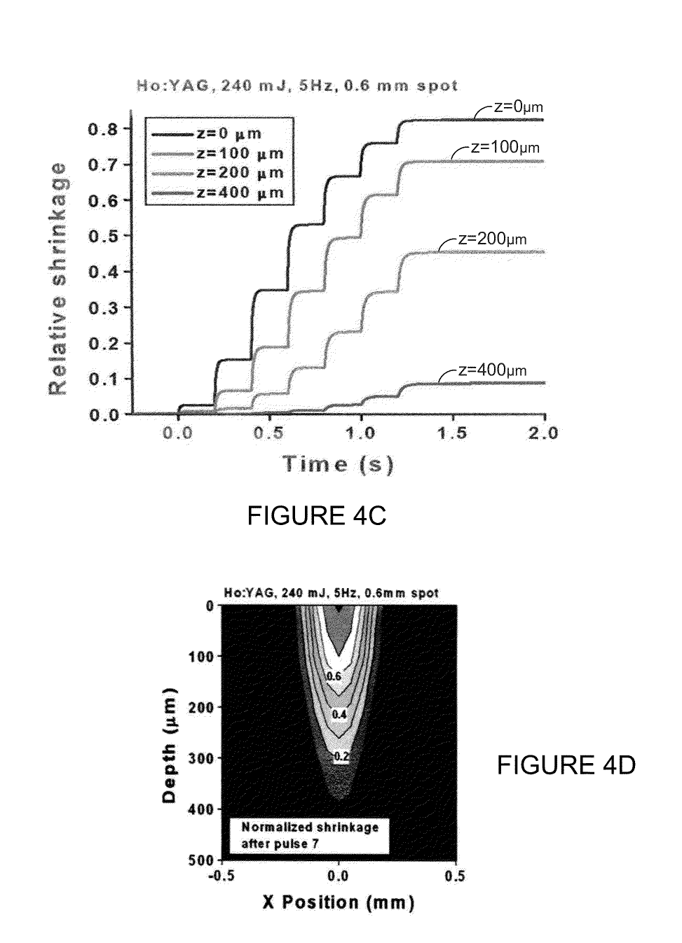

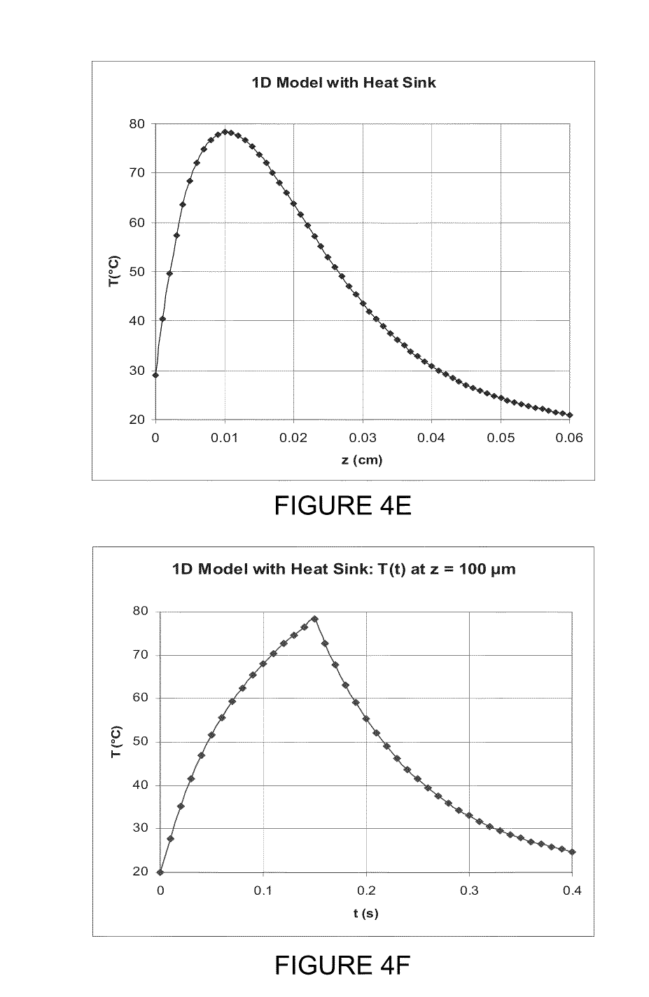

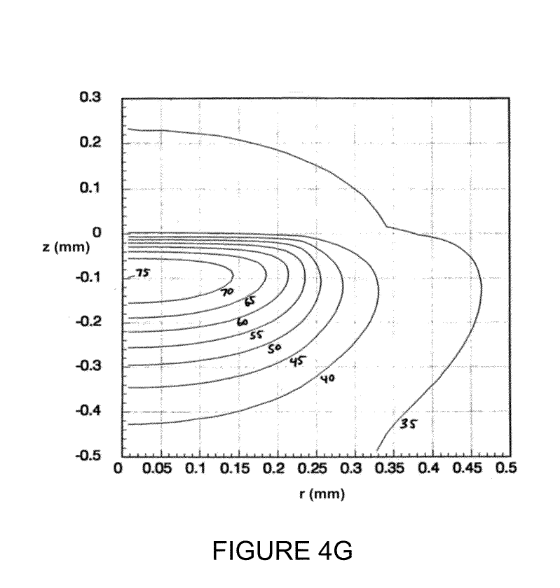

FIGS. 4A through 4G illustrate example temperature graphs showing how surgical eye procedures may or may not produce corneal tissue heating according to this disclosure. In particular, FIGS. 4A through 4D from Manns et al. (2002) illustrate how a conventional LTK procedure may produce stromal collagen shrinkage, while FIGS. 4E through 4G illustrate how an improved procedure may produce little or no stromal collagen shrinkage. The temperature graphs shown in FIGS. 4A through 4G are for illustration only.

Previous LTK treatments have been performed using a pulsed Ho:YAG laser operating at a wavelength of 2.13 μm. The treatments have involved seven pulses of laser irradiation at 240 mJ/pulse delivered onto eight spots of 0.6 mm diameter with a Gaussian irradiance distribution within each spot. Each spot therefore receives 30 mJ/pulse for each of seven pulses, or 210 mJ total energy. Individual pulses are approximately 200 μs in duration and are delivered at a pulse repetition frequency (PRF) of 5 Hz. Therefore, the sequence of pulses produces rapid heating within each 200 μs pulse, followed by cooling for 0.2 s, followed by the next pulse, until all seven pulses are delivered.

FIG. 4A illustrates the calculated corneal temperature T in the center of a laser-irradiated spot as a function of depth z into the cornea (where z=0 μm is the anterior epithelium) after each of seven laser pulses during a conventional LTK treatment. The first pulse produces a temperature increase of approximately 50° C. (from the assumed starting physiological temperature of 35° C.) at z=0 μm. Subsequent laser pulses produce additional temperature increases that are not as large since there is thermal relaxation (“cooling down”) between pulses and since the rate of thermal relaxation depends upon the initial temperature gradient (which increases as the tissue is heated further). If the laser pulses were continued beyond the seventh pulse and if the tissue was unchanged with respect to its thermal and optical properties, the tissue would reach equilibrium in which the heating caused by laser irradiation is balanced by the cooling caused by thermal relaxation.

FIG. 4B illustrates the calculated corneal temperature T in the center of a laser-irradiated spot at several depths (z=0, 100, 200, and 400 μm) as a function of time during the sequence of seven pulses during the conventional LTK treatment. The depth z=0 μm corresponds to the anterior epithelium. The depth z=100 μm corresponds to an anterior portion of the stroma (where stromal collagen shrinkage can occur). At each depth z, the corneal tissue is rapidly heated during each laser pulse, followed by a “cooling down” between pulses. The retained heat from previous pulse(s) adds to the temperature increase during the sequence of pulses.

FIG. 4C illustrates calculated “relative” stromal collagen shrinkage in the center of the laser-irradiated spot at several depths (z=0, 100, 200, and 400 μm) as a function of time during the sequence of seven pulses for the conventional LTK treatment. The calculated shrinkage is “relative” to a maximum value, stated to be “normalized” to a value of 0.35. In other words, a “relative” shrinkage of 100% corresponds to an actual length shrinkage of 35%.

FIGS. 4A through 4C consider only the center of each irradiated spot. The spots are often actually radially symmetric (axisymmetric), so the three-dimensional volume of treated tissue can be represented by a cross-section through the center of the volume in which the depth z and a radial coordinate x (the distance from the center of the spot) specify the full geometry. FIG. 4D illustrates a two-dimensional plot of the calculated “normalized” stromal collagen shrinkage after all seven laser pulses during the conventional LTK treatment. If the “normalized” stromal collagen shrinkage is integrated over the distribution shown in FIG. 4D, the actual fraction of stromal collagen that is shrunken within the entire treated volume of tissue is smaller than is indicated by FIG. 4C.

An improved optimal keratoplasty treatment may heat the cornea much less strongly than the prior procedure. The optimal keratoplasty treatment may use less energy per spot (such as 30 to 50 mJ/spot compared to 210 mJ/spot or greater). In addition, at least some (such as approximately half) of the optimal keratoplasty treatment energy may be conducted away from the cornea by a heat sink, such as the protective corneal applanator device 102. The optimal keratoplasty treatment therefore may cause less stromal collagen shrinkage than the conventional LTK treatment. Calculations reinforce this conclusion as follows.

In optimal keratoplasty treatments, the protective corneal applanator device 102 may reduce the starting cornea temperature to room temperature (such as approximately 20° C.). The protective corneal applanator device 102 may also provide an efficient heat sink to cool the cornea epithelium. An analytical one-dimensional (1D) model of a cornea in contact with a heat sink was used for parametric calculations below. The 1D model was also matched to more accurate numerical two-dimensional (2D) finite element calculations for similar irradiation conditions in order to improve calculated temperatures.

FIG. 4E illustrates the calculated corneal temperature T in the center of a laser-irradiated spot as a function of depth z into the cornea after 150 ms during an optimal keratoplasty treatment. This assumes that a continuous wave thulium fiber laser operates at a 1.94 μm wavelength (corresponding to a cornea absorption coefficient of 110 cm⁻¹) and irradiates a 600 μm diameter spot with uniform irradiance of 70 W/cm². Fresnel losses from the air/sapphire and sapphire/cornea interfaces (associated with a protective corneal applanator device 102 having a sapphire window) may reduce the irradiance to approximately 64 W/cm². For a 150 ms irradiation time, these conditions may lead to an energy density of approximately 30 mJ/spot on the proximal surface of the sapphire window and approximately 27 mJ/spot on the anterior surface of the cornea.

For the same irradiation conditions, FIG. 4F illustrates the calculated corneal temperature T in the center of the laser-irradiated spot at depth z=100 μm (at the peak temperature location for the 0.15 s irradiation shown in FIG. 4E) as a function of time. The temperature rises during laser irradiation up to t=0.15 s and then falls after irradiation is completed.

Taking a “worst case” scenario, FIG. 4F illustrates that corneal tissue is heated within the 70° C. to 80° C. range for approximately 0.06 s. Lower temperature regions may make a negligible contribution to stromal collagen shrinkage and thus do not provide clinically significant shrinkage. In this document, the phrase “clinically significant shrinkage” refers to shrinkage of corneal collagen that results in a noticeable change in a patient's vision. The amount of stromal collagen that remains unshrunken can be calculated from an exponential (first-order rate) equation, such as:

N(t)=N₀exp(−kt)

where N₀ is the starting amount of unshrunken collagen at time t=0, N(t) is the amount of unshrunken collagen at time t, and k is the collagen shrinkage rate coefficient. For T=70° C. to 80° C., the measured stromal collagen shrinkage rate coefficients by Borja et al. (2004) are k=0.008 to 0.015 s⁻¹. If the maximum rate coefficient of 0.015 s⁻¹ is used, the amount of unshrunken collagen calculated from this equation is 0.99895N₀, so only approximately 0.1% of the collagen at depth z=100 μm is shrunken.

Continuing the “worst case” scenario, FIG. 4E illustrates that a maximum thickness of approximately 120 μm of cornea stroma experiences the maximum heating (within the 70° C. to 80° C. range). This is the extent of the elevated temperature in depth along the centerline passing through the center of the irradiated spot. In the radial coordinate (perpendicular to the axial or depth coordinate), the temperature decreases towards the edge of the spot as shown in FIG. 4G for an irradiated cornea in contact with a sapphire heat sink. The 2D calculations represented in FIG. 4G are for a set of conditions similar to those used for the case represented in FIGS. 4E and 4F.

In FIG. 4G, the corneal tissue that is heated to a temperature of 70° C. or greater is similar to a disc or lozenge that has a radius of approximately 0.14 mm, with a thickness of approximately 120 μm. By inspection, the cross-sectional area of this disc represents less than 10% of the heated cross-sectional area of the full heated volume. The volume of tissue that is heated to a temperature of 70° C. or greater is smaller yet by a factor of approximately ten. As a result, less than 1% of the heated volume is at 70° C. or greater. Combining this volume estimate with the amount of collagen that is shrunken within the hottest volume (approximately 0.1%) leads to a conclusion that less than 0.001% of the stromal collagen within the heated corneal tissue volume may be shrunken using this procedure. Shrinkage at temperatures lower than 70° C. may be negligible for the short temporal duration of corneal tissue heating.

As a particular example, optimal keratoplasty treatment may be performed up to an energy density of approximately 40 or 48 mJ/spot (which could be approximately 36 or 43 mJ/spot, respectively, after taking Fresnel reflection losses into account) or more. More stromal collagen may be shrunken at these higher energy densities, but stromal collagen shrinkage may be a minor contributor to cornea shape changes even at this higher energy density.

Although FIGS. 4A through 4G illustrate example temperature graphs showing how surgical eye procedures may or may not produce corneal tissue heating, various changes may be made to FIGS. 4A through 4G. For example, other prior LTK or other eye procedures could differ from that shown in FIGS. 4A through 4D. Also, an optimal keratoplasty procedure could differ from that shown in FIGS. 4E through 4G.

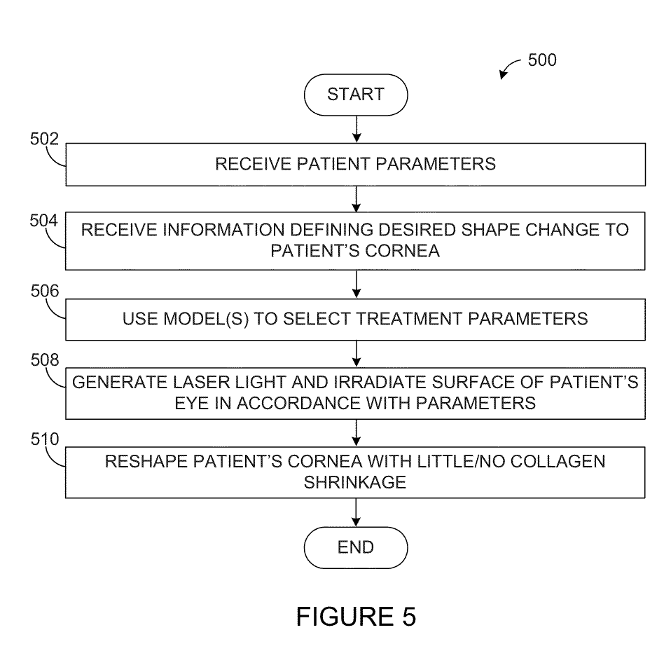

FIG. 5 illustrates an example method 500 for designing and implementing a cornea reshaping procedure according to this disclosure. The embodiment of the method 500 shown in FIG. 5 is for illustration only. Other embodiments of the method 500 could be used without departing from the scope of this disclosure.

A controller receives patient parameters at step 502. This could include, for example, a surgeon, nurse, or other personnel inputting the patient's age and other relevant factors into the controller 116. The controller 116 could also retrieve this data from other sources, such as electronic patient records.

The controller receives information defining the desired shape changes to the patient's eye at step 504. This could include, for example, the surgeon, nurse, or other personnel inputting information that defines the current and desired shapes of the patient's eye to the controller 116. This could also include the surgeon, nurse, or other personnel inputting information defining the changes to be made to the shape of the patient's cornea. The controller 116 could also retrieve or determine this data using information from other sources, such as information from a device that scans the patient's cornea and determines its current shape.

The controller uses one or more models to select the treatment parameters for the surgical eye procedure at step 506. This could include, for example, the controller 116 using one or more models 212 to determine how to achieve the desired shape changes to the patient's eye. Moreover, the controller 116 can select the parameters to achieve the desired shape changes while causing little or no stromal Type I collagen shrinkage in the patient's eye (thermal shrinkage or other modification of non-Type I collagen may or may not be permitted). As a particular example, as noted above, a significant amount of corneal stromal collagen (such as 10% or more) may shrink when heated to a temperature of at least 90° C. for a time of at least 10 s. As a result, the controller 116 can determine how to achieve the desired shape changes to the patient's eye without allowing the patient's corneal stromal collagen to be heated to or above this temperature for a time sufficiently long to cause significant shrinkage. In other words, shrinkage of corneal stromal Type I collagen can be avoided by not overheating the stroma for a lengthy period of time, based on the temperature-time history associated with a given laser irradiation or other heating. As a practical example, stromal collagen shrinkage could be avoided using continuous wave laser irradiation at 1.94 μm with 30 to 40 mJ/spot energy density for 150 ms. As a counterexample, Manns et al. (2002) have calculated that significant collagen shrinkage is produced when a pulsed Ho:YAG laser is used for LTK under historical, non-optimal treatment conditions.

Laser light (or non-laser light) is generated in accordance with these parameters and used to irradiate the patient's eye at step 508. This could include, for example, the controller 116 controlling the other components in the system 100 to control the irradiation of the patient's eye. As a result of the laser irradiation, the patient's cornea is reshaped with little or no stromal Type I collagen shrinkage at step 510. Because of this, problems with stromal collagen shrinkage, such as opacification, can be avoided.

Although FIG. 5 illustrates one example of a method 500 for designing and implementing a cornea reshaping procedure, various changes may be made to FIG. 5. For example, while described as using laser irradiation, any other suitable technique could be used to heat the tissue in the patient's cornea. Also, while described as being performed by the controller 116, various steps in FIG. 5 could be performed by another device, such as a computing device or other device configured to determine parameters for LTK or other eye procedures. In addition, while shown as a series of steps, various steps in FIG. 5 could overlap, occur in parallel, occur in a different order, or occur multiple times.

Returning to FIG. 1, as noted above, CXL is a surgical eye procedure for increasing corneal biomechanical strength by using ultraviolet or visible light (such as in the 300 nm to 450 nm spectral region) to increase the stiffness and strength of corneal tissue. A CXL procedure can involve the use of the system 100 in FIG. 1, such as when a light source 106 provides ultraviolet/visible light to the patient's eye via the corneal applanator device 102. Note that LTK and CXL procedures could occur independently or in combination together on the same patient's eye 104. For example, an optimal LTK procedure (described above) could treat certain portions of the patient's eye, and a CXL procedure could treat the same portions or different portions of the patient's eye. The CXL and optimal LTK procedures could be performed simultaneously or in any order. The CXL procedure could be performed in conjunction with an LTK procedure in order to help decrease regression of refractive correction induced by the LTK procedure. For instance, by stiffening the corneal tissue, this may allow the effects of the optimal LTK procedure to continue for longer periods of time (such as five or ten years or more) with less regression of corneal reshaping.

In a CXL procedure, ultraviolet or visible light initiates a photochemical reaction of excited riboflavin or photochemical products of excited riboflavin or that photo-sensitizes the generation of singlet oxygen, all of which can be reactive species that produce CXL. However, CXL in the standard “Dresden” protocol is highly invasive and uncomfortable for the patient. Briefly, the Dresden protocol involves removing the corneal epithelium over a large optical zone (7 mm to 9 mm diameter), soaking the cornea with a 0.1% riboflavin aqueous solution (typically with 20% Dextran) for thirty minutes, and then irradiating the riboflavin-soaked cornea with ultraviolet light at 365 nm for thirty minutes. The Dresden protocol has been used successfully to treat corneal disorders such as keratoconus and iatrogenic keratectasia caused by LASIK. Standard CXL using the Dresden protocol can stiffen human corneal tissue by over 300% with long-term stability, at least six years as judged by stabilization and reduction of keratoconus. However, there are many complications with this protocol. Standard CXL requires the complete removal of the epithelium for several days until re-epithelialization is complete, which is very uncomfortable. Moreover, this procedure requires one hour of treatment time, produces significant stromal haze, and has a relatively high complication rate (particularly for patients over 35 years old).

Some recent advances in CXL have included transepithelial delivery of riboflavin using benzalkonium chloride (BAK), a cytotoxic agent, to increase epithelial permeability to riboflavin and thereby eliminate removal of the epithelium. However, BAK may induce unwanted side effects, such as cytokine expression that triggers a corneal wound healing response. Also, some studies have indicated that the use of BAK does not promote delivery of riboflavin. Other recent advances have involved the use of RICOLIN TE from SOOFT or PARACEL from AVEDRO for transepithelial delivery of riboflavin. Further improvements in the delivery of riboflavin have included subepithelial channel or pocket formation by a femtosecond laser followed by subepithelial instillation of riboflavin, as well as iontophoresis of riboflavin across the corneal epithelium and into the corneal stroma.

Some aspects of the mechanism of action for riboflavin activated by ultraviolet light are clear. Ultraviolet A irradiation of riboflavin (at about 370 nm) causes electronic excitation of the riboflavin to an active species (RF*). RF* acts, in part, as a photo-sensitizer for the production of electronically excited molecular oxygen, O₂* (a¹Δ_{g}) termed “singlet oxygen”, by electronic energy transfer from RF* to ground electronic state oxygen (X³Σ_{g}⁻). Singlet oxygen initiates production of free radicals that cause cross-linking. However, there are some uncertain aspects for this mechanism of action. For example, RF* by itself may generate free radicals that cause cross-linking or may otherwise be degraded photo-chemically. Also, the exact nature of the corneal cross-links is under investigation, and they could involve several different types of cross-links between collagen, proteoglycan core proteins, and glycosoaminoglycans (GAGs).

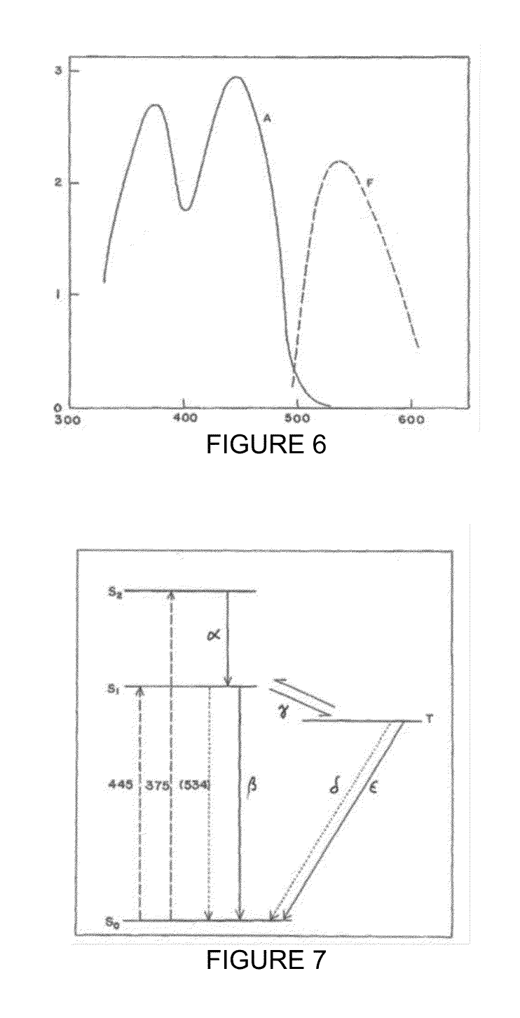

The electronic absorption (A) and fluorescence (F) spectra of riboflavin are shown in FIG. 6. An electronic energy level diagram of riboflavin is shown in FIG. 7. RF* has several fates as indicated by the transitions shown in FIG. 7, including:

F (labeled as 534 nm in FIG. 7): fluorescence with a peak wavelength at about 534 nm; the fluorescence quantum yield Φ_{F} is about 0.267 at pH=7, which means that about 27% of RF* radiates its energy and returns to the electronic ground state S₀ (but typically with some vibrational excitation that produces photo-thermal heating); however, if UVA light irradiance is high, this same molecule can absorb another photon, producing RF* again;

α (alpha) : rapid internal conversion from S₂ to S₁ (that leads to some vibrational excitation that produces photo-thermal heating); this process is very efficient (about 100% efficient) since the fluorescence spectrum is essentially identical following either S₂ or S₁ excitation;

γ (gamma): efficient intersystem crossing from S₁ to T (that leads to some vibrational excitation that produces photo-thermal heating); the triplet quantum yield Φ_{T} is about 0.54±0.07 in aqueous solution; this means that 54%±7% of RF* (either S₁ or S₂) yields triplet RF* that can, in turn, produce singlet oxygen O₂*(a¹Δ_{g}) (together with some vibrational excitation that produces photo-thermal heating); and

β (beta): considerable internal conversion from S₁ to S₀; this process has a quantum yield of about 0.19±0.07 if direct photochemistry (rather than photosensitization) associated with S₁ is small.

Other processes shown in FIG. 7 involving fates of triplet RF* either regenerate electronic ground state riboflavin by intersystem crossing δ (delta) (this process leads to some vibrational excitation that produces photo-thermal heating) or, with very low quantum yield, produces phosphorescence (ε). These processes do not affect energy disposition significantly. FIG. 7 does not show vibrational relaxation processes that occur in solution or in corneal tissue. For example, both internal conversions are immediately followed by vibrational relaxation that heats riboflavin and its surroundings.

Working through the energetics, for each molecule of riboflavin that is excited by one photon of 370 nm light, a maximum of about 66% of the absorbed photon energy is channeled into simple photo-thermal heating of riboflavin and its surroundings (water and/or corneal tissue). In other words, riboflavin acts like a simple chromophore for photo-thermal heating of, for example, corneal stromal tissue. At low irradiance (3 mW/cm²) used in standard CXL, heating effects are not important. At higher irradiance (such as above 10 W/cm²), photo-thermal heating effects that lead to photo-thermal keratoplasty may contribute significantly to tissue changes.

For a cornea without riboflavin, the absorption coefficient of light at 365 nm wavelength is essentially zero. For riboflavin at 0.1% concentration in the anterior stroma of an eye, the absorption coefficient is about 58 cm⁻¹, and the absorption is due to the riboflavin. The “effective” absorption coefficient for photo-thermal keratoplasty is about 66% of this value, or 39 cm⁻¹.

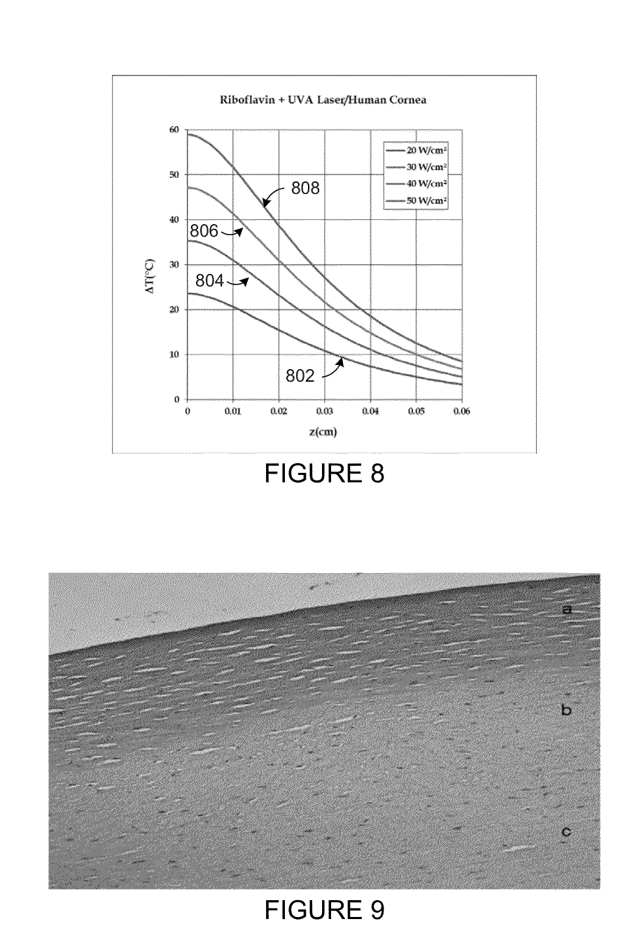

FIG. 8 shows exact one-dimensional (1D) calculations of temperature rise in corneal stromal tissue using this “effective” absorption coefficient. The temperature rises are represented by lines 802 (20 W/cm²), 804 (30 W/cm²), 806 (40 W/cm²), and 808 (50 W/cm²). The depth “z” shown in FIG. 8 is for the corneal stroma only and is in cm units (where 1 cm=10 ⁴ μm). The corneal epithelium does not absorb significant riboflavin and hence is not heated by a UVA laser. However, on the timescale of continuous wave (CW) laser irradiation (which may be in the range of 1 to 1,000 milliseconds), thermal diffusion into the epithelium (not shown in FIG. 8 but at a depth of −0.005 cm to 0 cm) and into an optional applanation window takes place, so the temperature rise is decreased. A more elaborate bioheating model calculation can be performed, but the net effect is that the anterior-most stroma is most strongly cooled by thermal diffusion, leading to a temperature rise maximum that is about 5 μm to 100 μm deep within the stroma.

The temperature rises shown in FIG. 8 are comparable to those produced using the optimal LTK procedure described above and would likely cause corneal reshaping. Hence, 0.1% riboflavin acts as an absorber dye to cause photo-thermal effects, and this photo-thermal effect is in addition to the photo-sensitizer effect of RF* that produces CXL. The two effects (photo-thermal and photo-sensitizer) would likely amplify each other when they occur simultaneously; for instance, CXL efficiency may increase at higher temperature.

The histology of corneal tissue treated by standard CXL reveals various similarities between CXL and optimal LTK tissue effects. FIG. 9 shows a photomicrograph of porcine corneal tissue after standard CXL (in which the corneal epithelium is completely removed). In region “a”, the tissue is compressed as indicated by darker staining, and there are many vacuoles (presumably containing water). This pattern of tissue compression and vacuole formation is the same as or very similar to that observed for porcine tissue following optimal LTK treatment (see treatment spots 350 and 352 in FIG. 3B). The intermediate depth stroma (region “b”) is a transition zone, and the deepest stroma (region “c”) is a normal but edematous zone that is untreated but swollen.

Another similarity between CXL-treated corneas and optimal LTK-treated corneas is that CXL also produces corneal flattening. This flattening is caused by compression of corneal stroma tissue (due to either CXL or optimal LTK treatment). It might be possible to achieve enhanced compression by treating the stroma using both CXL and optimal LTK treatments. If enhancement (and hence larger effect) can be obtained, different sequences of treatments can be examined to identify the largest enhancement (CXL followed by optimal LTK; optimal LTK followed by CXL; simultaneous CXL and optimal LTK). Note that simultaneous CXL and optimal LTK may permit the use of lower energy densities during optimal LTK to achieve a desired corneal reshaping effect.

In accordance with one aspect of this disclosure, a “minimalist” approach toward using CXL is provided as an adjunctive therapy to improve the efficacy (including the duration of effect) of LTK procedures (such as the optimal LTK procedure described above). The basic elements of “minimalist” CXL include:

administration of riboflavin or other photo-sensitizer(s) to the cornea, possibly along with at least one additional dye to increase absorption of light for CXL and photo-thermal heating of corneal stroma;

LTK treatment of the cornea (before, during, or after CXL); and

UV or visible CXL treatment of the cornea (to cause “curing” of the LTK-treated tissue).

As example alternatives, a UV or visible light source can be operated at a suitably high irradiance so that the corneal tissue receives both CXL and LTK treatment simultaneously, or both the LTK and UV/visible light sources can be combined to irradiate the corneal tissue simultaneously (such as at high irradiance like 10 W/cm²).

The riboflavin or other photo-sensitizer(s) can be administered in an appropriate dose in a specified area of the corneal stroma (such as the anterior-most 50-100 μm of the corneal stroma) and in an appropriate pattern (such as within the 5 mm to 8 mm optical zones where LTK is performed). In particular embodiments, administration of riboflavin in a formulation (such as RICOLIN TE from SOOFT or PARACEL from AVEDRO) could permit rapid transepithelial (TE) delivery without significant side effects. Ideally, the photo-sensitizer may be acceptable as a “natural” reagent that does not require regulatory approval by the U.S. Food and Drug Administration and/or other agencies as a “new drug” (although regulated drugs could also be used).

For the UV or visible “curing”, instead of broadbeam irradiation of the whole cornea, effective UV/visible light for CXL can be delivered through the optical fibers in the array 110 to the same corneal locations that are irradiated during the LTK procedure. Previous CXL treatments have been performed using a very large corneal area (in the full 7 mm to 9 mm optical zone), which would be counterproductive for optimal LTK since corneal flattening occurs in the center of this large corneal area, typically by about two Diopters. When optimal LTK is used to correct hyperopia by corneal steepening, initial corneal flattening would make hyperopia correction more difficult. To achieve enhancement of optimal LTK effects by CXL, both treatments can be localized in the same or similar areas so that corneal flattening (due to stromal compression) occurs additively. This can also help to prevent damage to the cornea outside of the treatment spots, such as by minimizing keratocyte apoptosis and endothelial cell damage in thin corneas. In addition, since optimal LTK can produce a unique pattern of corneal multi-focality (as described below), substantially the same treatment spots can be used for CXL in order to preserve that corneal multi-focality.

Simultaneous use of both the CXL and LTK light sources or use of the CXL light source at a suitably high irradiance to produce both CXL and LTK effects may be advantageous from the standpoint of causing increased corneal reshaping effect, as well as longer duration of effect, compared to using the two light sources sequentially. This is due to the fact that CXL may depend upon temperature. For example, the efficiency and extent of CXL may be improved at the increased temperature caused by photo-thermal heating.

A light source 106 can miniaturized by using a low power laser (such as a He—Cd laser operating at a 325 nm wavelength or a diode laser operating at a 375 nm, 395 nm, or 405 nm wavelength). The beam can follow the same optical path through the system 100 to the patient's eye as the treatment beams used during LTK. The combined LTK/CXL system enables rapid treatment of a patient's cornea in a single procedure (without unnecessary and potentially harmful treatment of tissue surrounding the LTK-treated spots or line segments).

Ideally, the CXL treatment can extend the effectiveness and duration of effect of the LTK treatment by “sealing” or “stabilizing” the change(s) in corneal structure and function caused initially by LTK. CXL may also produce an enhancement of the initial LTK effect. The LTK-treated stroma may behave like a sponge that has had water expressed from it by the photo-thermal effect produced by LTK irradiation. Photo-thermal processing of stromal tissue may also alter other extracellular matrix components of the stroma such as proteoglycans. Over time, the treated stromal tissue may reach homeostasis by restoration of its original hydration state and, possibly, by restoration of proteoglycan (PG) conformation(s). CXL may retard the rate(s) at which these restoration processes occur, thereby reducing regression of LTK effects.

A further improvement to CXL adjunctive therapy involves the use of deuterated water (D₂O) as a solvent for riboflavin or other photo-sensitizer(s). Deuterated water improves the efficiency of action of singlet oxygen by reducing its solvent quenching. Application of deuterated water in LTK procedures is discussed in more detail in U.S. Pat. No. 7,691,099.

In the above description of combining LTK and minimal CXL procedures, it was described that the CXL light could be provided to the same spots or line segments that are irradiated during the LTK procedure. In some embodiments, the LTK treatment is performed only within discrete spots or line segments in the paracentral and peripheral regions of the cornea (for example, within the 5 mm to 8 mm optical zone). Thus, the CXL radiation could be limited to use in those same areas. Any suitable source of visible or ultraviolet wavelength light can be used in the CXL procedure, such as a low power laser like a continuous wave or pulsed diode laser operating at 375 nm, 395 nm, or 405 nm. As an alternative, the light can be produced by an incoherent light source that irradiates a larger area but that is restricted to irradiating only discrete treatment spots or line segments by use of an optical mask. In general, any light source that generates light effective in causing CXL can be used. Note that both UVA light sources operating at about 365 nm and visible light sources operating at about 436 nm have been effective in inducing CXL and corneal “stiffening” in riboflavin-soaked porcine eyes. One particular example of a light source is a violet GaN diode laser operating at 405 nm, which is currently available with output power levels that match combined photo-thermal/photo-sensitizer requirements.

Moreover, any suitable technique could be used to deliver the riboflavin or other photo-sensitizer(s) to a patient's cornea. This can include iontophoresis, forming a “pocket” under the corneal epithelium, injection with micro-needles, and other techniques for drug delivery. Since the cross-linking photo-sensitizer(s) can diffuse radially and axially from the initial delivery position, the initial delivery position may be displaced from the CXL treatment location. For example, the initial delivery position may be very peripheral, such as outside the 8 mm optical zone.

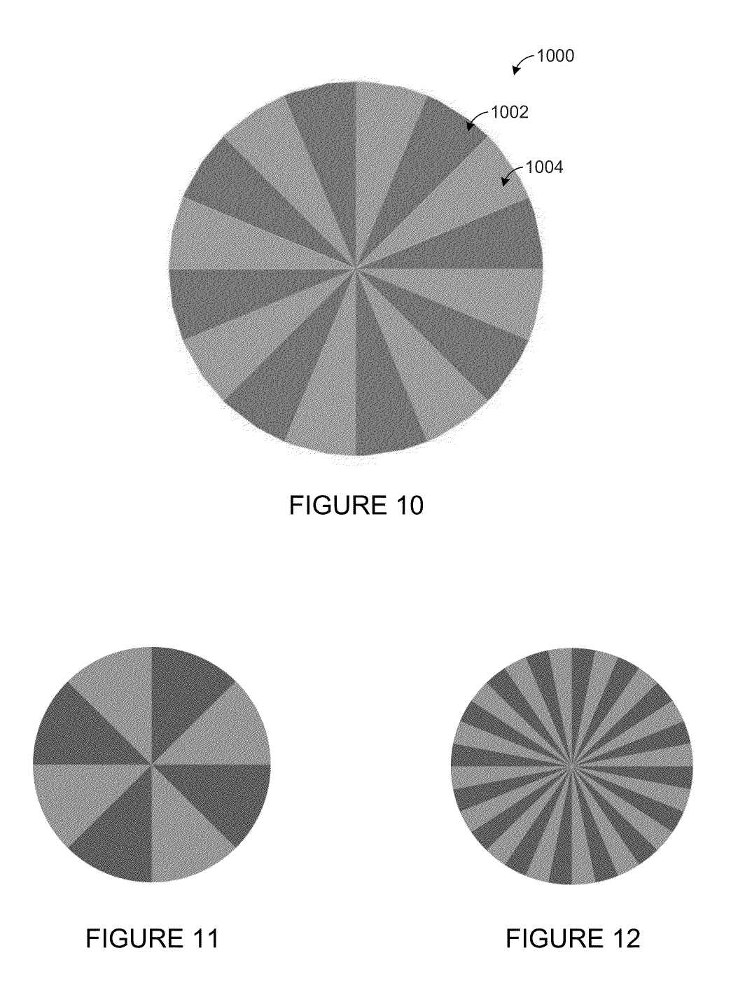

In addition, the ordinary pattern of CXL over the entire cornea is known to cause corneal thinning and net corneal flattening. This type of treatment pattern may be suitable to reduce myopia and to reduce conical protrusion of keratoconus, but it may be unsuitable for use in causing central corneal steepening and with the optimal LTK treatment pattern (such as an eight-leaf rosette of alternating flatter and steeper sectors as shown in FIG. 10) that provides corneal multi-focality.

FIGS. 10 through 12 illustrate example multi-focal refraction patterns that can be used during a corneal cross-linking (CXL) or other procedure or in optical devices according to this disclosure. Numerous patterns of refraction variation have been used to provide ocular multi-focality in eyewear (spectacles and contacts), intraocular lenses, and laser ablation patterns. These patterns are typically used to allow subjects with inadequate accommodation to focus on objects that are at variable distances (far, intermediate, and near). Examples of these patterns of refraction variation include bifocal and progressive lenses in eyewear, “premium” multi-focal intraocular lenses, and central near (central “island”) ablation patterns in laser-assisted in situ keratomileusis (LASIK).

In many applications (such as providing high quality of both distance and near visual acuity), these patterns of refraction variation have proven inadequate. For example, four corneal shapes produced by laser ablation [global optimum for curvature and asphericity (GO), central steep island (CSI), decentered steep island (DSI), and centered steep annulus (CSA)] have been investigated as candidate patterns of refraction variation. All have been shown to be non-optimal for corneal compensation for presbyopia (see, for example, Koller et al., “Four corneal presbyopia corrections: Simulation of optical consequences on retinal image quality”, J. Cataract Refract. Surg., 2006, pp. 2118-2123). Current corneal shapes used in PresbyLASIK (i.e., LASIK to overcome symptoms of presbyopia by pseudo-accommodation) include central near (e.g., CSI) and central far patterns with or without asphericity variations. These corneal shapes are typically axisymmetric about the corneal vertex or the pupil center. All of these PresbyLASIK patterns of refraction variation have deficiencies, such as loss of the quality of vision as pupil size and/or illumination vary. Some of these patterns of refraction variation are also associated with unwanted ocular aberrations that produce vision disturbances such as glare and halo.

In accordance with this disclosure, improved or optimal patterns of refraction variation can be used on any optical surface. The optical surfaces could include eyewear such as spectacles and contact lenses, intraocular lenses, and anterior and posterior surfaces of the cornea and the crystalline lens (the phrase “optical device” refers to a physical device having an optical surface but does not include any part of a human body). The optimal patterns of refraction variations can be used to improve visual acuity for objects at far, intermediate, and near distances while preserving high quality of vision (such as contrast sensitivity and stereoacuity) and minimizing or eliminating ocular aberrations that produce vision disturbances (such as glare and halo). One example of an optimum pattern of refraction variation is shown in FIG. 10. In particular, FIG. 10 illustrates a multi-focal pattern 1000 of alternating sectors 1002-1004 of greater (1002) and lesser (1004) refractive powers. The sectors 1002-1004 could, for instance, have alternating steeper (1002) and flatter (1004) curvatures of the anterior surface of the cornea.

The multi-focal pattern 1000 may be spherical or aspheric (with respect to the radial coordinate). Also, while there are eight sectors 1002 and eight sectors 1004 in FIG. 10, the multi-focal pattern 1000 may have different numbers of alternating sectors 1002-1004 (see FIGS. 11 and 12 for other examples). The multi-focal pattern 1000 may further be axisymmetric with respect to the visual axis or some other axis such as the line of sight. In addition, the multi-focal pattern 1000 may be tailored to be non-axisymmetric in order to further optimize visual acuity and the quality of vision. For example, a tailored corneal shape may compensate for aberrations of the crystalline lens of the eye, such as lenticular astigmatism.

The multi-focal pattern 1000 may be produced on the anterior surface of the cornea by photo-thermal treatment, photo-ablation, or other surgical eye procedure. Also, the multi-focal pattern 1000 may be produced on other ocular surfaces, such as the posterior surface of the cornea, the anterior and/or posterior surface of the crystalline lens, or the anterior and/or posterior surface of an intraocular lens using any suitable surgical procedure. Further, the multi-focal pattern 1000 may be produced on objects, such as the anterior and/or posterior surface of eyewear (like spectacles and contact lenses). In addition, the multi-focal pattern 1000 may be produced within optical elements by, for example, refractive index variation.

In FIGS. 10 through 12, the various sectors may represent equal or unequal angular regions, and the sectors may have variable angular widths from the center to the periphery of the cornea. The sectors may also have “graduated” changes within each angular range. These patterns can be considered extensions of the pattern of regular astigmatism (appropriate to a single cylindrical lens) that produces the “conoid of Sturm” already known to provide useful corneal multi-focality to the eye. Lenticular astigmatism may also provide useful lenticular multi-focality to the eye.

These multi-focal patterns could be used during an optimal LTK corneal reshaping procedure to provide multi-focality for a patient. CXL can also be used to help prevent regression of the LTK-induced corneal reshaping.

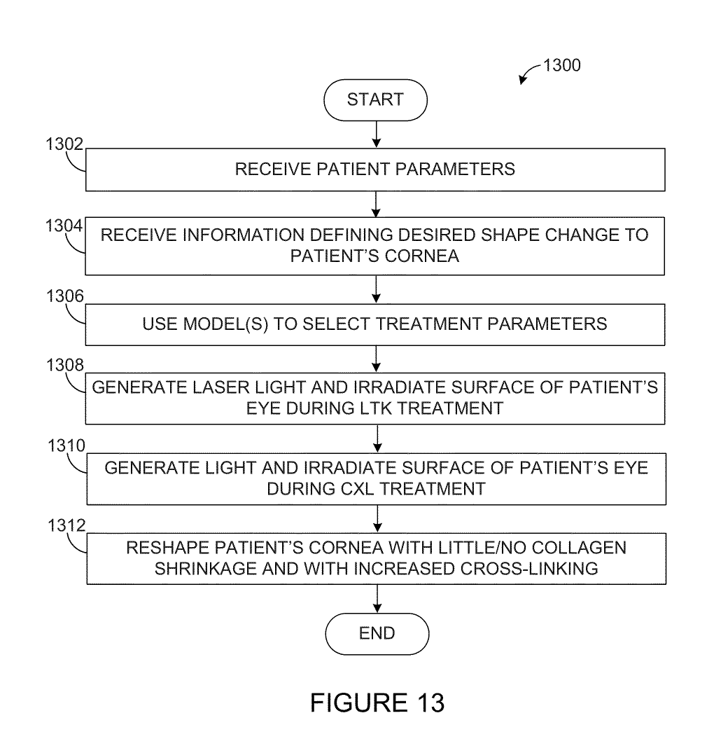

FIG. 13 illustrates an example method 1300 for designing and implementing a cornea reshaping procedure involving LTK and CXL according to this disclosure. The embodiment of the method 1300 shown in FIG. 13 is for illustration only. Other embodiments of the method 1300 could be used without departing from the scope of this disclosure.

A controller receives patient parameters at step 1302. This could include, for example, a surgeon, nurse, or other personnel inputting the patient's age and other relevant factors into the controller 116. The controller 116 could also retrieve this data from other sources, such as electronic patient records.

The controller receives information defining the desired shape changes to the patient's eye at step 1304. This could include, for example, the surgeon, nurse, or other personnel inputting information that defines the current and desired shapes of the patient's eye to the controller 116. This could also include the surgeon, nurse, or other personnel inputting information defining the changes to be made to the shape of the patient's cornea. The controller 116 could also retrieve or determine this data using information from other sources, such as information from a device that scans the patient's cornea and determines its current shape.

The controller uses one or more models to select the treatment parameters for the surgical eye procedure at step 1306. This could include, for example, the controller 116 using one or more models 212 to determine how to achieve the desired shape changes to the patient's eye during an LTK procedure. As described above, the controller 116 can select the parameters to achieve the desired shape changes while causing little or no stromal Type I collagen shrinkage in the patient's eye (thermal shrinkage or other modification of non-Type I collagen may or may not be permitted). This step also includes identifying treatment parameters for a CXL procedure. As noted above, the LTK and CXL procedures could occur simultaneously or in any order, and the parameters can vary depending on how the LTK and CXL procedures occur.

Laser light (or non-laser light) is generated in accordance with these parameters and used to irradiate the patient's eye during an LTK procedure at step 1308. This could include, for example, the controller 116 controlling the other components in the system 100 to control the irradiation of the patient's eye during the LTK procedure. Light is also generated in accordance with these parameters and used to irradiate the patient's eye during a CXL procedure at step 1310. This could include, for example, the controller 116 controlling the other components in the system 100 to control the irradiation of the patient's eye during the CXL procedure after riboflavin or other photo-sensitizer has been applied to the patient's eye. Note that the photo-sensitizer can be applied to the patient's eye before or after the LTK procedure has occurred.

As a result of the irradiation, the patient's cornea is reshaped with little or no stromal Type I collagen shrinkage and improved cross-linking at step 1312. Because of this, problems with stromal collagen shrinkage, such as opacification, can be avoided. Moreover, the cross-linking can help to reduce the amount of regression in the patient's eye.

Although FIG. 13 illustrates one example of a method 1300 for designing and implementing a cornea reshaping procedure involving LTK and CXL, various changes may be made to FIG. 13. For example, while described as using laser irradiation, any other suitable technique could be used to heat the tissue in the patient's cornea. Also, while described as being performed by the controller 116, various steps in FIG. 13 could be performed by another device, such as a computing device or other device configured to determine parameters for LTK or other eye procedures. Further, while described as supporting LTK and CXL procedures, the method 1300 could involve CXL and some other type of cornea reshaping procedure. In addition, while shown as a series of steps, various steps in FIG. 13 could overlap, occur in parallel, occur in a different order, or occur multiple times. As a particular example, the order of the LTK and CXL procedures could be reversed, or the LTK and CXL procedures could occur at the same time.

In some embodiments, various functions described above are implemented or supported by a computer program that is formed from computer readable program code and that is embodied in a computer readable medium. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

It may be advantageous to set forth definitions of certain words and phrases used throughout this patent document. The terms “application” and “program” refer to one or more computer programs, software components, sets of instructions, procedures, functions, objects, classes, instances, related data, or a portion thereof adapted for implementation in a suitable computer code (including source code, object code, or executable code). The terms “include” and “comprise,” as well as derivatives thereof, mean inclusion without limitation. The term “or” is inclusive, meaning and/or. The phrase “associated with,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. The term “controller” means any device, system, or part thereof that controls at least one operation. A controller may be implemented in hardware or in hardware with firmware or software. It should be noted that the functionality associated with any particular controller may be centralized or distributed, whether locally or remotely.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. A system comprising:
at least one radiation source configured to generate radiation for a cornea reshaping procedure and a corneal cross-linking procedure;
a delivery device configured to deliver the radiation to the patient's eye; and
a controller configured to control the delivery of the radiation to the patient's eye during the cornea reshaping procedure and the corneal cross-linking procedure.

2. The system of claim 1, wherein the controller is configured to control the delivery of radiation so that the cornea reshaping procedure and the corneal cross-linking procedure at least partially overlap in time.

3. The system of claim 1, wherein:
the delivery device is configured to deliver radiation to specified areas of the patient's eye during the cornea reshaping procedure; and
the delivery device is configured to deliver radiation only to the same specified areas of the patient's eye during the corneal cross-linking procedure.

4. The system of claim 1, wherein the radiation during the corneal cross-linking procedure follows a common optical path as the radiation during the cornea reshaping procedure.

5. The system of claim 1, wherein the at least one radiation source comprises:
a visible, infrared, or near-infrared source for the cornea reshaping procedure; and
an ultraviolet or visible source for the corneal cross-linking procedure.

6. The system of claim 1, further comprising:
optical fibers configured to transport the radiation from the at least one radiation source to the delivery device.

7. The system of claim 1, wherein the delivery device comprises a protective corneal applanator device.

8. The system of claim 1, wherein the controller is configured to control the delivery of radiation during the cornea reshaping procedure to create alternating sectors of greater and lesser refractive powers in the patient's eye.

9. The system of claim 1, wherein the system is configured to deliver radiation having a power of at least about 10 W/cm² to the patient's eye.

10. An apparatus comprising:
at least one memory configured to store treatment parameters for a cornea reshaping procedure and a corneal cross-linking procedure; and
at least one processing device configured to control at least one radiation source in order to control delivery of radiation to a patient's eye during the cornea reshaping procedure and the corneal cross-linking procedure.

11. The apparatus of claim 10, wherein the at least one processing device is further configured to select the treatment parameters using at least one model.

12. The apparatus of claim 10, wherein the at least one processing device is configured to control the delivery of radiation so that the cornea reshaping procedure and the corneal cross-linking procedure at least partially overlap in time.

13. The apparatus of claim 10, wherein:
the at least one processing device is further configured to control a translation stage that determines which of multiple optical fibers direct the radiation towards the patient's eye; and
the at least one processing device is configured to control the translation stage so that radiation is delivered to specified areas of the patient's eye during the cornea reshaping procedure and radiation is delivered only to the same specified areas of the patient's eye during the corneal cross-linking procedure.

14. The apparatus of claim 10, wherein the at least one processing device is configured to control the at least one radiation source by controlling delivery of power to the at least one radiation source.

15. The apparatus of claim 10, wherein the at least one processing device is configured to control the delivery of radiation during the cornea reshaping procedure to create alternating sectors of greater and lesser refractive powers in the patient's eye.

16. The apparatus of claim 10, further comprising:
one or more controls configured to receive input data associated with the cornea reshaping procedure and the corneal cross-linking procedure; and
a display configured to provide output data associated with the cornea reshaping procedure and the corneal cross-linking procedure.

17. A method comprising:
generating radiation for a cornea reshaping procedure and a corneal cross-linking procedure;
delivering the radiation to a patient's eye; and
controlling the delivery of the radiation to the patient's eye during the cornea reshaping procedure and the corneal cross-linking procedure.

18. The method of claim 17, wherein controlling the delivery of the radiation comprises controlling the delivery of the radiation so that the cornea reshaping procedure and the corneal cross-linking procedure at least partially overlap in time.

19. The method of claim 17, wherein delivering the radiation to the patient's eye comprises:
delivering radiation to specified areas of the patient's eye during the cornea reshaping procedure; and
delivering radiation only to the same specified areas of the patient's eye during the corneal cross-linking procedure.

20. The method of claim 17, further comprising:
applying a solution of deuterated water and a photo-sensitizer to the patient's eye prior to delivering the radiation to the patient's eye during the cornea reshaping procedure and the corneal cross-linking procedure.

21. An optical device comprising:
an optical surface comprising alternating angular regions of greater and lesser refractive powers.
